(19) United States
(12) Patent Application Publication
(10) Pub. No.: US 2006/0015127 A1
(43) Pub. Date: Jan. 19, 2006

(54) Injection drill assembly

(75) Inventor: Wen-Kwei Chien, Taichung City (TW)

Correspondence Address: BACON & THOMAS, PLLC, 625 SLATERS LANE, FOURTH FLOOR, ALEXANDRIA, VA, 22314, US

(73) Assignee: I CHU SPRING CO., LTD., Taichung City (TW)

(21) Appl. No.: 10/889,082

(22) Filed: Jul. 13, 2004

(51) Int. Cl. A61B 17/32

(52) U.S. Cl. 606/167; 604/264

(57) ABSTRACT

An injection drill assembly for an intra-osseous injection apparatus includes a needle, a needle hub, and a clamp. The needle hub is made of a flexible material of low rigidity, including a first section, a second section located behind the first section, and a tunnel longitudinally running therethrough. The first section has two annular ridges formed respectively at two ends thereof, and a body portion between the two annular ridges. Alternatively, the needle hub has a thin sidewall. The needle is inserted through the tunnel of the needle hub. The clamp is fitted onto the body portion of the needle hub to squeeze and tightly clamp the first section of the needle hub to further fixedly clamp the needle.

### BACKGROUND OF THE INVENTION

1. Field of the Invention

The present invention relates generally to medical apparatuses, and more particularly to an injection drill assembly for an intra-osseous injection apparatus.

2. Description of the Related Art

A conventional intra-osseous injection apparatus, which is commonly used in drilling bones or teeth and further delivering an anesthetic or other fluids into other parts of a living body, either human or animal, includes a drill member 100 and a driving mechanism (not shown). The drill member, as shown in FIG. 1, has a needle 110 and a sleeve 120. The sleeve 120 has a tunnel 121 longitudinally running therethrough and a hole 122 located at a peripheral wall thereof for communicating with the tunnel 121. The needle 110 is inserted into the sleeve 120 and an adhesive, such as ultraviolet cured resin or hot glue, is put into the hole to fill space between the needle 110 and the sleeve 120, such that the needle 110 and the sleeve 120 are fixedly connected with each other. However, the adhesive has to be sticky enough to fix the needle 110 to the sleeve 120 such that the production cost is increased. In addition, if the intra-osseous injection apparatus is operated for a long time or in high-temperature environment, the adhesive is subject to degeneration to incur separation of the needle 110 from the sleeve 120.

### SUMMARY OF THE INVENTION

The primary objective of the present invention is to provide an injection drill assembly for an intra-osseous injection apparatus; the injection drill assembly is structurally tight to prevent a needle from separation from a needle hub.

The secondary objective of the present invention is to provide an injection drill assembly for an intra-osseous injection apparatus; the injection drill assembly can be automatically assembled by machine to enhance the production efficiency.

The foregoing objectives of the present invention are attained by the injection drill assembly, which includes a needle, a needle hub, and a clamp. The needle hub includes a first section, a second section located behind the first section, and a tunnel longitudinally running therethrough. The first section has two annular ridges formed respectively at two ends thereof, and a body portion between the two annular ridges. The needle is inserted through the tunnel of the needle hub. The clamp is fitted onto the body portion of the needle hub to squeeze and tightly clamp the first section of the needle hub to further fixedly clamp the needle.

### BRIEF DESCRIPTION OF THE DRAWINGS

FIG. 1 is a perspective view of the prior art;

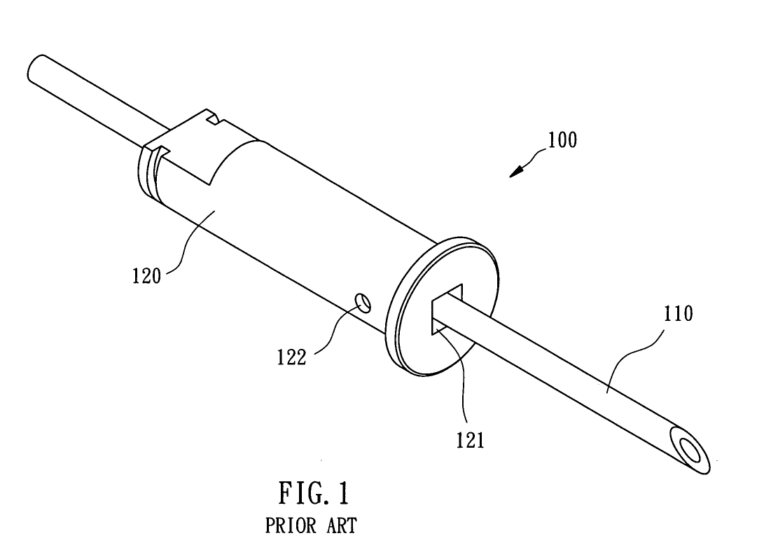

FIG. 2 is an exploded view of a first preferred embodiment of the present invention;

FIG. 3 is a perspective view of the first preferred embodiment of the present invention;

FIG. 4 is an exploded view of a second preferred embodiment of the present invention;

FIG. 5 is a perspective view of the second preferred embodiment of the present invention; and

FIG. 6 is a cross-sectional view taken from a line 6-6 indicated in FIG. 5.

### DETAILED DESCRIPTION OF THE INVENTION

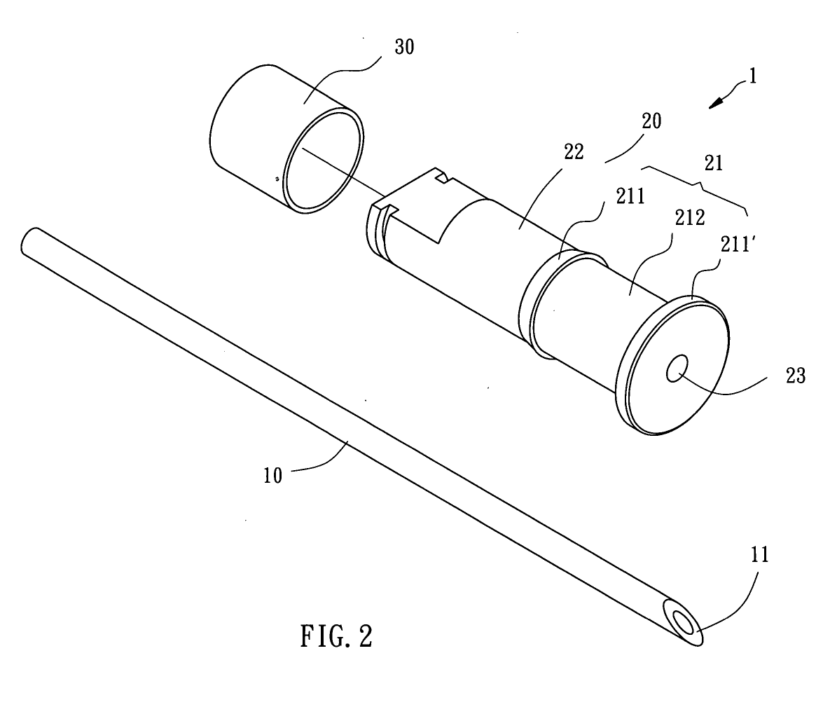

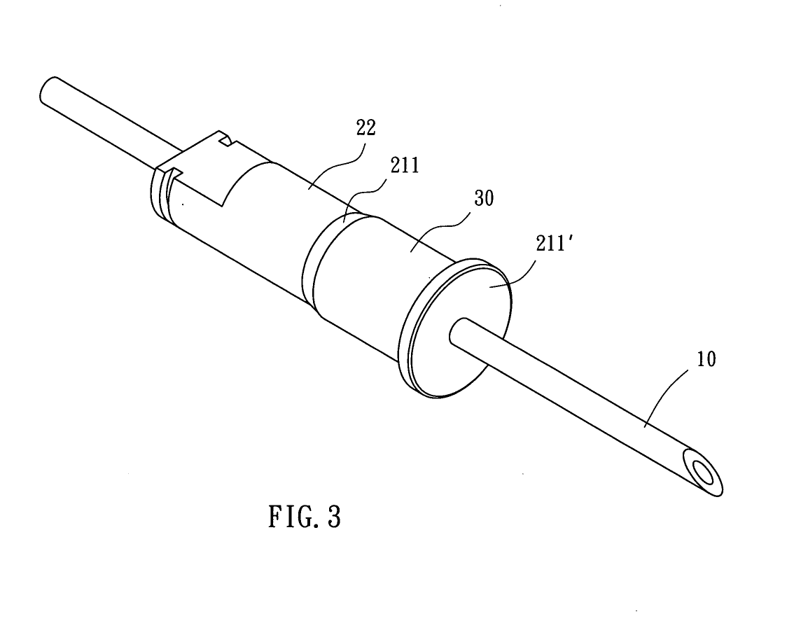

Referring to FIGS. 2 and 3, an injection drill assembly 1 constructed according to a first preferred embodiment of the present invention includes a needle 10, a needle hub 20, and a clamp 30.

The needle 10 is an elongated hollow member, having a beveled cutting end 11 at a front end thereof for delivering an anesthetic into the human or animal body while drilling through any of the bones or teeth.

The needle hub 20 is an elongated hollow member made of a flexible material, which has a low rigidity, including a first section 21, a second section 22 located behind the first section 21, and a tunnel 23 longitudinally running through the first and second sections 21 and 22 for inserting the needle 10. The tunnel 23 has the same diameter as the needle 10 for tightly inserting the needle 10. The first section 21 has two annular ridges (first annular ridge 211 and second annular ridge 211′) formed at two ends thereof, and a body portion 212 located between the two annular ridges 211 and 211′. The annular ridge 211 is tapered to have a decreasing diameter from a front end thereof to a rear end thereof. The diameter of the rear end of the annular ridge 211 is identical to the second section 22 to facilitate the clamp 30 to pass through the annular ridge 211. The second section 22 can be connected with an intra-osseous injection apparatus (not shown) for synchronic rotation with the intra-osseous injection apparatus (not shown). Alternatively, the body portion 212 has a thin sidewall.

The clamp 30 is a hollow member made of a flexible material, such as plastic, having an inner diameter, which is slightly smaller than an outer diameter of the body portion 212, smaller than that of the front end of the annular ridges 211 and that of the annular ridge 211′, and larger than that of the second section 22, and having the same length as the body portion 212. The clamp 30 is fitted onto the body portion 212 to tightly squeeze and clamp the body portion 212 of the first section 21 of the needle hub 20 and is fixedly located between the two ridges.

While assembling the injection drill assembly 1, insert the needle 10 through the tunnel 23 of the needle hub 20, and then put the clamp 30 to pass through the second section 22 and the annular ridge 211 abutting the second section 22 and to further be fitted onto the body portion 212 between the two ridges 211 and 211′ of the needle hub 20. Because the clamp 30 is made of flexible material and the annular ridge 211 is tapered, the clamp 30 can slightly deformably pass through the ridge 211 abutting the second section 22 though the diameter of the front end of the ridge 211 is larger than the inner diameter of the clamp 30. Because the inner diameter of the clamp 30 is smaller than the outer diameter of the body portion 212 and the needle hub 20 is made of the material of low coefficient of elasticity, low rigidity, and small moment of inertia of area, when the clamp 30 is fitted onto the body potion 212, the body potion 212 can be deformably compacted by the clamp 30 to further tightly clamp the needle 10. The inner diameter of the clamp 30 is smaller than that of the front end of the ridges 211 and that of the ridge 211′ of the needle hub 20, such that when the clamp 30 is fitted onto the body portion 212, the two ridges 211 and 211′ can prevent the clamp 30 from moving out of the needle hub 20 to further keep the clamp 30 fixedly tightly clamping the body portion 212 of the needle hub 20.

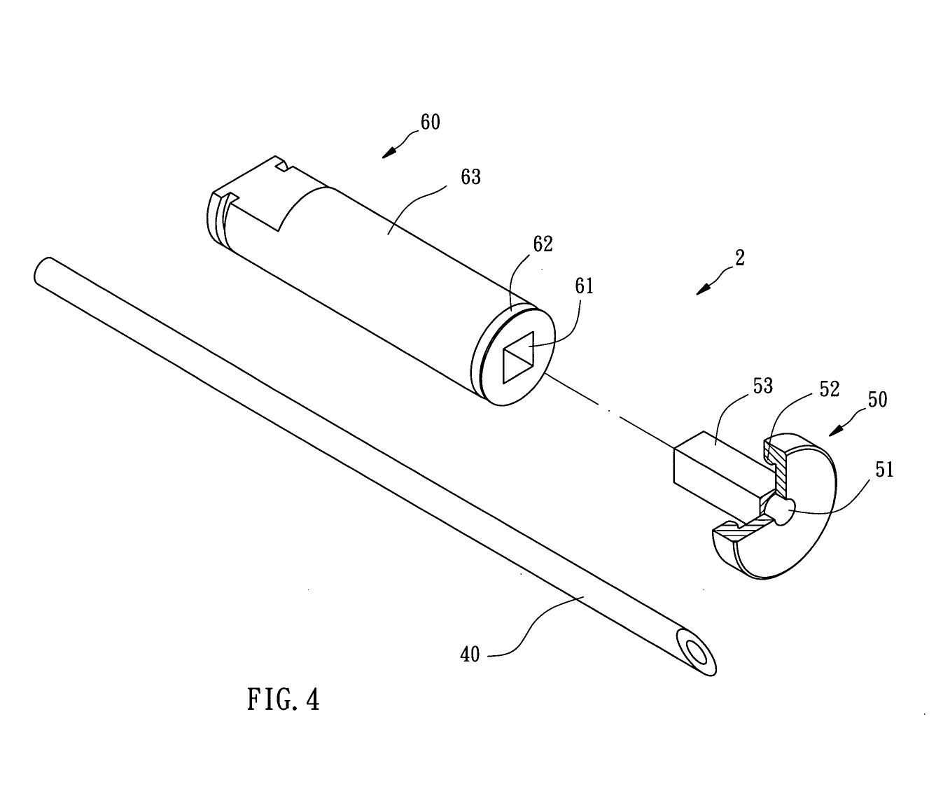

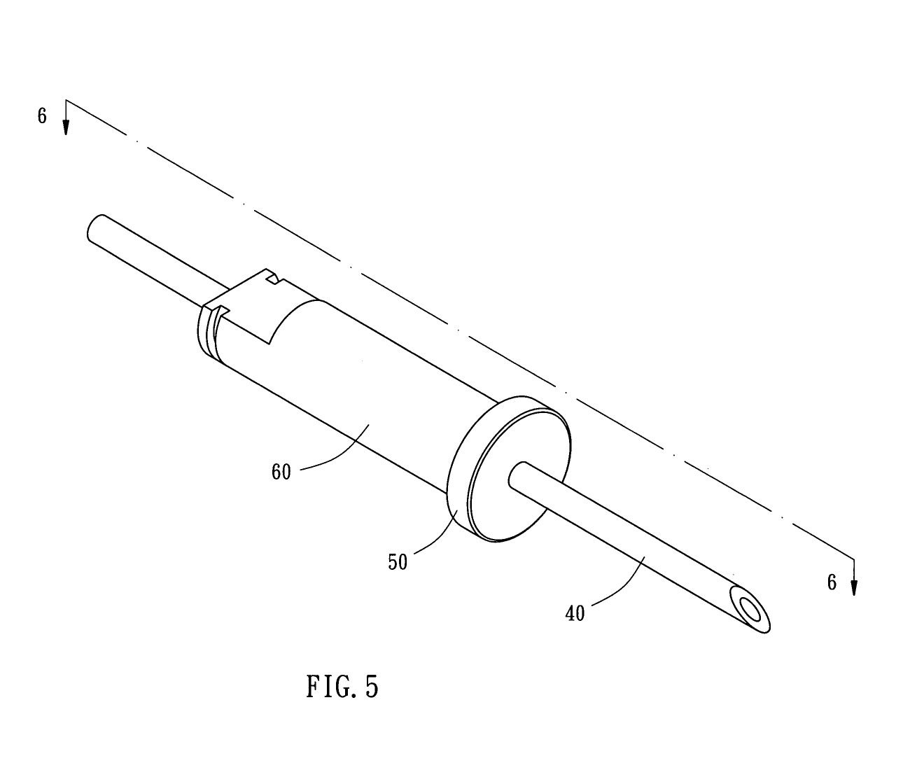

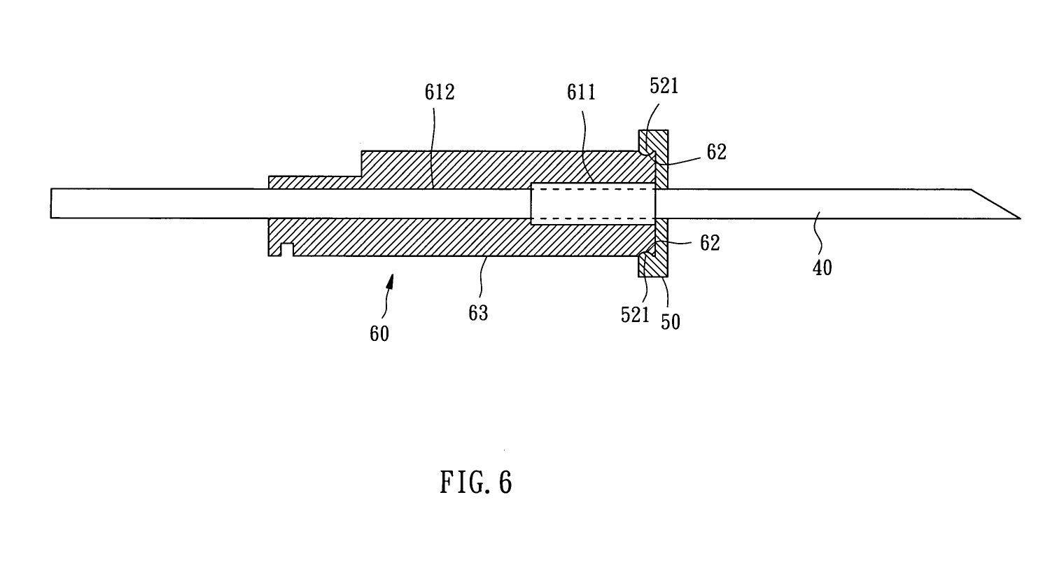

Referring to FIGS. 4-6, the injection drill assembly 2 constructed according to a second preferred embodiment of the present invention includes a needle 40, a needle base 50, and a needle hub 60.

The needle 40 is substantially identical to that of the aforementioned preferred embodiment.

The needle base 50 is a hollow member made of flexible material, which has a low rigidity, having a first tunnel 51 running therethrough for inserting the needle 40, a head portion 52, and a body portion 53 formed behind the head portion 52. The first tunnel 51 has a diameter identical to that of the needle 40 for tightly inserting the needle 40. The body portion 53 is non-circular tubular member, which cross-section can be triangle, quadrangle, or other polygon. The head portion 52 has an annular wedge portion 521, which outer diameter is larger than that of the head portion 53. Alternatively, the body portion 53 has a thin sidewall.

The needle hub 60 is an elongated hollow member made of flexible material, such as plastic, having a second tunnel 61 running therethrough for inserting the needle 40, an annular incision 62 formed at a front end thereof for engaging the annular wedge portion 521 of the needle base 50, and a main body 63 for connected with the intra-osseous injection apparatus (not shown). The second tunnel 61 is provided with a first section 611 and a second section 612 located behind the first section 611. The second section 612 has a diameter identical to that of the needle 40 for tightly inserting the needle 40. The first section 611 is tapered to have a decreasing diameter from a front end thereof to a rear end thereof. The diameter of the rear end of the first section 611 is larger than that of the second section 612 and smaller than that of the body portion 53. The diameter of the front end of the first section 611 is identical to that of the body portion 53 of the needle base 50. The first section 611 has a non-circular cross-section, which can be triangle, quadrangle, or other polygon corresponding to that of the body potion 53 for ensuring synchronic rotation of the needle base 50, the needle hub 60 and the needle 40, being as long as the body portion 53.

While assembling the injection drill assembly 2, insert the needle 40 through the first tunnel 51 to be tightly jammed with the needle base 50, and then insert the needle base 50 into the second section 612 of the second tunnel 61 to enable the wedge portion 521 to engage the annular incision 62 of the needle hub 60. While needle base 50 is inserted into the needle hub 60, the body portion 53 of the needle base 50 can be deformably squeezed by the first section 611 to be further tightly fitted into the first section 611 of the second tunnel 61 of the needle hub 60, such that the needle 40 can be fixedly clamped. When the injection drill assembly 2 is rotated together with the intra-osseous injection apparatus (not shown), the needle base 50 will not separate from the needle hub 60 and keep rotation together with the needle hub 60.

In addition, the above procedures of the assembly are accomplished by machine, such that present invention can be automatically produced without manual checkout to further enhance the production efficiency.

What is claimed is:

1. An injection drill assembly for an intra-osseous injection apparatus, said injection drill assembly comprising:
a hollow needle;
a needle hub made of a flexible material of low rigidity, said needle hub having a first section, a second section located behind said first section, and a tunnel running therethrough for inserting said needle, said first section having two annular ridges formed respectively at its two ends, and a body portion located between said two annular ridges; and
a clamp made of a flexible material and fitted onto said body portion of said needle hub to squeeze said body portion, whereby said body portion is deformably squeezed inwards to further fixedly clamp said needle.

2. The injection drill assembly as defined in claim 1, wherein said tunnel of said needle hub has the same diameter as an outer diameter of said needle for tightly inserting said needle; each of said two annular ridges has a diameter larger than an inner diameter of said clamp for stopping said clamp from moving; said body portion of said needle hub comprises a diameter larger than an inner diameter of said clamp.

3. The injection drill assembly as defined in claim 1, wherein said body portion comprises a thin sidewall.

4. The injection drill assembly as defined in claim 2, wherein said two annular ridges comprises a first annular ridge and a second annular ridge, said first annular ridge abutting said second section of said needle hub and being tapered to have a decreasing diameter from a front end thereof to a rear end thereof; each of said needle hub and said clamp is made of plastic.

5. The injection drill assembly as defined in claim 4, wherein said body portion comprises a diameter smaller than that of a front end of said first annular ridge.

6. An injection drill assembly for an intra-osseous injection apparatus, said injection drill assembly comprising:
a hollow needle;
a needle base made of a flexible material of low rigidity, said needle base having a first tunnel running therethrough for inserting said needle, and a head portion having an annular wedge portion, a body portion formed behind said head portion; and
a needle hub made of a flexible material and having a second tunnel running therethrough for inserting said needle, and an annular incision formed at a front end thereof for engaging said annular wedge portion of said needle base, said second tunnel having a first section and a second section, said first section being tapered to have a decreasing diameter from a front end thereof to a rear end thereof, the diameter of the rear end of said second section of said second tunnel being larger than that of said second section and smaller than that of the body portion, the diameter of the front end of said second section of said second tunnel being identical to that of said head portion of said needle base.

7. The injection drill assembly as defined in claim 6, wherein said body portion of said needle base comprises a non-circular cross-section; said first section of said second tunnel of said needle hub comprises a non-circular cross-section corresponding to that of said body potion and a length identical to that of said head portion

8. The injection drill assembly as defined in claim 7, wherein said first tunnel of said needle base has the same diameter identical as that of said needle for tightly inserting said needle; said body portion of said needle base comprises a polygonal cross-section; said second section of said second tunnel of said needle hub has a diameter identical to that of said needle for tightly inserting said needle.

9. The injection drill assembly as defined in claim 6, wherein said body potion comprises a thin sidewall.

10. The injection drill assembly as defined in claim 6, wherein each of said needle base and said needle hub is made of plastic.
